(19) United States
(12) Patent Application Publication
(10) Pub. No.: US 2006/0100507 A1
(43) Pub. Date: May 11, 2006

(54) Method for evaluation of medical findings in three-dimensional imaging, in particular in mammography

(76) Inventor: Thomas Mertelmeier, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/256,045

(22) Filed: Oct. 21, 2005

(30) Foreign Application Priority Data
Oct. 21, 2004 (DE) 10 2004 051 401.1

(51) Int. Cl. A61B 5/05

(52) U.S. Cl. 600/425

(57) ABSTRACT

In a method for medical evaluation in three-dimensional imaging, in particular in mammography, projection exposures of a subject to be examined are generated and are stored in electronic form. Slice images are reconstructed from the projection exposures with a reconstruction method. A physician reviews and medically evaluates the slice images and marks a positive medical finding in the slice image with a first marker. A CAD system medically evaluates the slice images and marks a positive medical finding in the slice image with a second marker. A slice image with a first marker and a second marker deviating from one another is reconsidered and reevaluated by the physician.

### BACKGROUND OF THE INVENTION

1. Field of the Invention

The present invention concerns a method for evaluation of medical findings in three-dimensional imaging, in particular in mammography.

2. Description of the Prior Art

Imaging methods are of increasing importance in medical technology. The subject of the examination is a human or animal body. Classical methods such as, for example, the x-ray radiology, in which two-dimensional images of a body region to be examined are generated, have existed for a long time. In addition to this are modern methods, such as nuclear magnetic resonance tomography and computed tomography, which also generate three-dimensional images or, respectively, approximate, quasi-three-dimensional images (via slice representation).

In digital tomosynthesis as is known, for example, from DE 198 42 944 A1, 3D information that is provided in the form of reconstructed slices is acquired with a digital projection x-ray system.

In practice, classical tomography (known as “slicing”) has proved its value as a precursor to digital tomosynthesis in many applications. Digital tomosynthesis is currently under evaluation, primarily in examinations of lungs, joints and the female breast.

The acquisition of 3D information does in fact increase the information content acquired about the examined body region, but the physician conducting the examination must also evaluate a significantly larger data quantity in the form of a multiple slice exposures. Such imaging examination methods therefore are not easy for many physicians to evaluate (with regard to the effort expended to make a medical finding) for screening purposes, thus presenting difficulties to accomplish serial examination of patients with a patient throughput of, for example, more than 50 people per day.

### SUMMARY OF THE INVENTION

An object of the present invention is to improve the ability to make a medical finding in three-dimensional imaging, in particular in mammography.

This object is achieved in accordance with the invention by a method for medical diagnosis in three-dimensional imaging, in particular in mammography, in which projection exposures of a subject to be examined are generated and are stored in electronic form. Slice images are reconstructed from the projection exposures with a reconstruction method. A physician considers and evaluates the slice images and marks a positive medical finding in a slice image with a first marker. A CAD system evaluates the slice images and marks a positive medical finding in the slice image with a second marker. A slice image with first marker and second marker deviating from one another is reconsidered and reevaluated by the physician.

Slice images are two-dimensional images that are reconstructed from a number of projection exposures in the tomosynthesis of a subject to be examined. Suitable reconstruction methods of the type known from computed tomography (CT)) are used for this purpose. The subject to be examined is a body region of a human or animal, in particular a female breast examined in a mammography screening. Due to the electronic storage of the corresponding projection exposures and reconstructed slice images, these can easily be processed with all of the usual advantages of electronic data storage and processing.

The physician examining the subject considers and evaluates a slice image in the usual manner.

If, based on his or her professional experience and expertise, the physician establishes a positive medical finding in the slice image, for example a lesion in the form of a tumor or a micro-calcification, the physician marks the suspicious point in the slice image with a first marker. This ensues, for example, by clicking the corresponding image region on the screen of a computer on which the slice image is shown, and by electronic storage of the first marker at the corresponding point.

From the large number of slice images available, the physician can quickly select one or more exemplars assessed by him or her as particularly informative or interesting upon cursory review and only evaluate or medically assess these selected images, or the physician can evaluate all slice images. For example, one or two particularly promising slice images can be presented to the physician by an automatic image processing system. A corresponding slice image for further processing in the medical finding method is characterized by the electronic marking with the first marker.

In addition to the physician, a computer-aided detection or diagnostic system (CAD system) medically evaluates the slice images. All slice images or only a specific selection thereof can be medically evaluated in turn. Comparable to the physician, the CAD system searches for positive medical findings in the slice images by corresponding image processing algorithms (for example based on an expert system or the like) and, if applicable, marks these with a second marker.

For the subject to be examined, the entire set of slice images acquired from the subject are now available, the slice images being by a first marker or a second marker at specific points in the case of a positive medical finding by the physician or the CAD system.

In a further method step, the slice images are now examined for the existence of the first marker or the second marker. The following possibilities can result.

Slice exposures which have neither a first marker nor a second marker are characterized as non-critical (i.e. without medical finding) and are not evaluated further.

Alternatively, slice exposures can exhibit a first marker and second marker at the same point (generally within the resolution of the localization precision of the medical finding method that is used). This means that both the physician and the CAD system have marked the corresponding image region as a positive medical finding, thus the diagnoses of the physician and the CAD system agree. This image region is unambiguously assessed as a positive medical finding and likewise does not need to be evaluated further.

As a third possibility, either a first marker or a second marker but not both simultaneously can occur in a specific image region. The medical findings of the physician and the CAD system thus deviate from one another for such an image region, so a corresponding slice image is presented to the physician again for a final medical evaluation. The ultimate decision to make a medical finding for the appertaining image region is incumbent on the physician. The physician can either maintain his or her positive finding or revise it when the CAD system has not assessed or has negatively assessed, the corresponding image region, or the physician can re-check an image region appearing as suspect to the CAD system (positive medical finding of the CAD system) and revise or approve the CAD medical finding.

Overall computer support in the medical evaluation of the multiple slice images is offered to the physician by the method, such that these positive findings overlooked by the physician but detected by the CAD system are brought to the physician's attention, while the physician's positive findings are confirmed for safety or are questioned by the CAD system. Overall, the medical finding assurance of the physician is increased. For the physician this represents a significant saving in work, effort or time, primarily in the case in which the physician medically evaluates only a small number (thus not all) of the slice images available. Serial examinations in the framework of a screening are thus significantly accelerated, and the patient throughput is increased. The detection rate of positive medical findings is increased by the increased sensitivity of the method relative compared to the sole evaluation by a physician. Fluctuations in the detection precision of positive findings that may occur for the same physician (fitness on a particular day, concentration, fatigue) or between different physicians (different level of experience, training, subjective evaluation measures) are reduced (thus various medical findings are more comparable), primarily in screening, by the use of a neutral, independent, automatic CAD medical evaluation method.

The probability of false positive medical findings is increased if the physician is required to evaluate a large number of slice images in a given time frame. By the consideration of fewer slice images by the physician in accordance with the invention, the diagnosis is accelerated, the physician is unburdened, the patient throughput is increased and thus the productivity of the physician is increased.

Due to the number of slice images to be assessed, serial examinations are possible at all only for tomosynthesis since the physician does not have to assess all slice images.

For medical evaluation, the CAD system can use correlations between slice images of adjacent slices in the subject. For this purpose, known 3D filter algorithms for 2D slices are used in which computer-aided systems are advantageous in a known manner. The detection rate for positive medical findings thus is increased for the CAD system since it can utilize more information than the physician by the correlation with adjacent slices. Correlations between the individual slice images are primarily larger in tomosynthesis than in other slice methods such as, for example, CT, but which correlations cannot be used, or can be used only with great difficulty by a human observer.

A tomosynthesis method can be used as a reconstruction method. The subject must only be irradiated from fewer directions than given convention reconstruction methods. The patient is exposed to less radiation.

As an alternative or in addition to the correlations between slice images, the CAD system can use the projection exposures for analysis or making a medical finding (detection, diagnosis, classification) and their correlation with the reconstructed slice images. This increases the reliability of the computer analysis.

A slice image that corresponds to a mammography exposure in a conventional form can be reconstructed from projection exposures by the reconstruction method. The physician can consider and medically evaluate the mammography exposure instead of the projection exposures. This is possibly more familiar to the physician than the assessment of slice images, so the physician can more quickly or more confidently arrive at a positive or negative medical finding of the mammography exposure. The finding work is made easier for the physician, so that the task can be finished in a shorter time. The CAD system furthermore medically assesses slice images, projection exposures or the mammography exposure and marks positive medical findings therein with second markers. Positively assessed images (thus images provided with a second marker) are presented to the physician. As explained above, the physician thus is given the opportunity to revise his or her medical finding or to find support for it.

Instead of slice images, the CAD system can medically assess the reconstructed thick slice (conventional mammography image) and mark a positive finding in the image with a second marker. If the physician also assesses the same image, and the physician and CAD system operate with this image as an initial basis, the checking of the agreement of first and second markers can also be automated again.

The CAD system can be integrated into the reconstruction method. The integration is followed, for example, by the use of corresponding reconstruction filters. An image region with positive medical finding by the CAD system is then emphasized in the image. An image similar to as in classical mammography is thus presented to the physician, but the appertaining region positively assessed by the CAD system is already emphasized and thus immediately comes to the physician's attention. Moreover, under the circumstance the physician can more simply and therewith more quickly arrive at a distinct medical finding by the emphasis.

Both the physician and the CAD system can also mutually medically assess both the slices images and the projection exposures. The CAD system thus conducts a double assessment, whereby its probability of a match or conclusion precision for positive or negative medical findings is furthermore increased.

Slice images of various slice thickness can be generated or reconstructed with the reconstruction method. Slice images of various slice thicknesses can then be presented to the physician and the CAD system. For example, a slice image of a specific slice thickness is magnetic resonance examination suitable for review and visual assessment by the physician than for automatic assessment of the image content by the CAD system. Both slice images can be optimally evaluated in this manner. The match precision by the physician and CAD system in the medical evaluation is thereby increased.

### DESCRIPTION OF THE DRAWINGS

FIG. 1 schematically illustrates a system for computer-aided medical evaluation in three-dimensional imaging in accordance with the invention.

FIG. 2 is a workflow diagram for a method for computer-aided medical evaluation in three-dimensional imaging in accordance with the invention.

FIGS. 3a and 3b substantially illustrate markings in slice images for explaining the invention.

### DESCRIPTION OF THE PREFERRED EMBODIMENTS

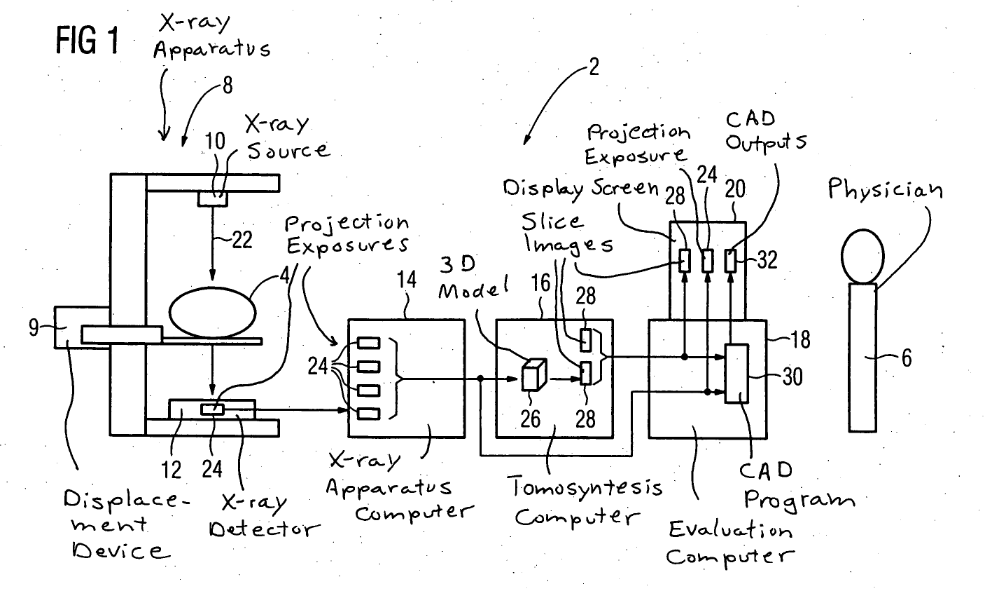

FIG. 1 shows a tomosynthesis device 2 for medical evaluation in three-dimensional imaging, i.e., in the framework of a tomosynthesis, with a patient 4 to be examined and a physician 6 conducting the examination. The tomosynthesis device 2 has an x-ray apparatus 8 with an x-ray source 10, a mechanical displacement device 9 and a digital x-ray detector 12 and a computer 14 belonging to the x-ray apparatus 8, a tomosynthesis computer 16 and a medical evaluation computer 18 with a screen 20.

The examination of the patient 4, for example the mammography of a female breast, is initiated at what is known as the acquisition workstation, in the form of the computer 14 which serves as a workstation for the medical personnel or the physician 6. The patient 4 is irradiated by x-rays 22 emanating from the x-ray source 10 and individual projections or projection exposures 24 are generated in the detector 12. The patient 4 and/or the x-ray source 10 and detector 12 are successively varied in terms of their position relative to one another by the mechanical displacement device 9. It is hereby possible to move either the patient 4 or the x-ray apparatus (or its components) or both together. These projection exposures 24 so generated are then transferred to the computer 14 and stored there.

The projections 24 are subsequently transmitted to the tomosynthesis computer 16 which generates a three-dimensional model 26 of the exposed region of the patient 4 via a digital tomosynthesis method from the two-dimensional data of the projection exposures 24. This model 26 is in turn composed of arbitrary slice images 28 which, for example, are slice images through the irradiated body region of the patient 4, which slice images cannot be shown solely by the x-ray technique, or representations in the form of classical mammography exposures.

The individual projections 24 and the slice images 28 are transmitted to the medical evaluation computer 18 and selectively displayed on the screen 20 where they can be observed by the physician 6. Moreover, in the medical evaluation computer 18 they are supplied to a CAD process, thus a CAD program 30 in the form of a computer program running therein.

The projections also already can be shown at the acquisition workstation 14, for example for checking the examination subjects (for example patient positioning or image quality). The tomosynthesis or reconstruction computer 16 and the CAD or medical evaluation computer 18 can be formed by a single computer.

The outputs 32 of the CAD program 30 are likewise shown on the screen 20. The physician 6 conducts the medical evaluation of the patient 4 on the medical evaluation computer 18 by evaluating the projections 24, the slice images 28 and the outputs 32.

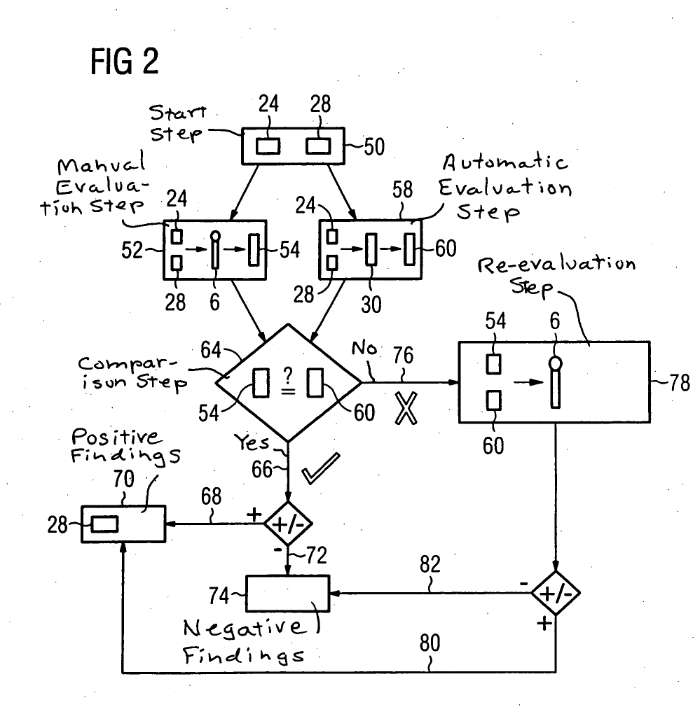

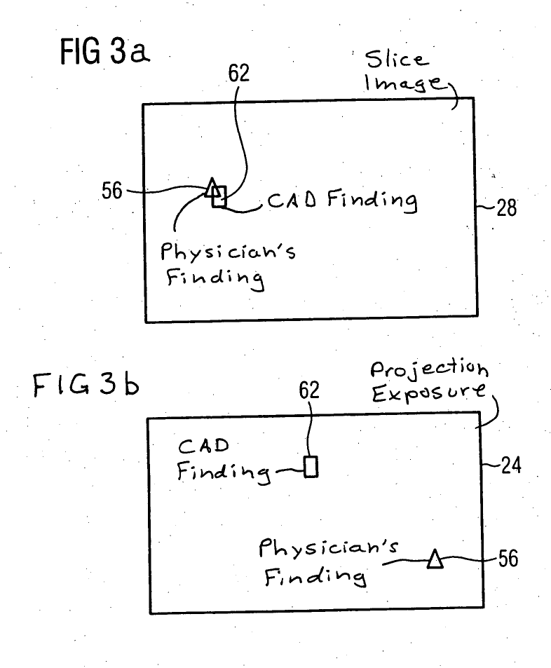

The workflow of the method for medical evaluation in three-dimensional imaging or, respectively, in the framework of a tomosynthesis is shown in a workflow diagram in FIG. 2. As described above, the projection exposures 24 and slice images 28 are generated in a start step 50 in or after the irradiation of the patient 4. All or only individual projection exposures 24 or slice images 28 are presented to the physician 6 on the screen 20 in a classical (manual) evaluation step 52, whereupon the physician conducts a medical evaluation of the image material and arrives at a physician's finding 54. For example, as indicated in FIG. 3a the physician marks a suspicious point in the slice image 28 with a finding marker 56. The physician optionally likewise marks an image point in a projection exposure 24 with a finding marker 56, as shown in FIG. 3b. The physician suspects malignant lesions in the body of the patient 4 at the points of the finding markers 56.

Simultaneously with the classical evaluation step 52, the CAD program 30 conducts an automatic medical evaluation of this image contents on the same or a different selection of slice images 28 and projection exposures 24 in an automatic evaluation step 58 and arrives at a CAD finding 60. In the CAD finding 60, the CAD program 30 marks with a finding marker 62 the same point as the physician 6 in the slice image 28 of FIG. 3a, since there it detects a malignant lesion. In the single-slice exposure 24 shown in FIG. 3b, it likewise marks a different point than was marked by the physician 6 with a finding marker 62.

In a comparison step 64, the medical findings 54 and 60 are compared. The positions of the finding markers 56 and 53 are compared in the medically evaluated slice images 28 and 24 of FIGS. 3a and 3b. In the slice image 28 of FIG. 3a, the finding markers 56 and 62 lie at the same point (within the resolution of the finding precision), and thus mark the same appertaining body region of the patient 4 as the location of a malignant lesion, which represents a positive medical finding. Since the physician's finding 54 and the CAD finding 60 agree in this regard, a YES decision 66 is made and the appertaining slice image 28 is associated with the set of the positive findings 70 via the positive branch 68, which means that the patient 4 exhibits a positive finding at the point detectable on the slice image 28.

If a slice image 28 (not shown) were to exhibit no finding markers 62 and 56 (and thus physician's finding 54 and CAD finding 60 again agree), this would likewise lead to a YES decision 66, but supplied to the negative set 74 via the negative branch, meaning that the patient 4 is characterized as unambiguously without a medical finding.

If, in the comparison step 64, physician's finding 54 and CAD finding 60 differ because different image points have been marked (as indicated in FIG. 3b), the NO decision 76 is made and a re-evaluation step 78 ensues.

In the re-evaluation 78, the physician is presented with his or her physician's finding 54 and the CAD finding on the screen 20, for example the single-slice exposure 24 with various locations of finding markers 56 and 62 in the example of FIG. 3b. The physician checks the medical finding by a renewed, precise study of the slice image 28 and possible evaluation of secondary material such as a slice image 28 of greater slice thickness resembling a conventional mammographic exposure. As a result of this re-evaluation, the physician may conclude that he or she had correctly marked a point in the slice image 28, which in fact did not represent a medical finding, with a finding marker 56, and thus the physician removes this incorrect marker 56. The physician alternatively may conclude, based on the use of other information, that the point marked in the slice image 28 by the CAD system 30 with the finding marker 62 does in fact indicate a valid positive medical finding that was not marked by the physician. The slice image 28 of the FIG. 3b is therefore likewise associated with the positive set 70 via the positive branch 80. If, in the re-evaluation step 78, the physician were to conclude that his or her negative final medical finding is, in fact, correct, the slice image 28 would be associated with the negative set 74 corresponding to the negative branch

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for medically three-dimensional images of an examination subject, comprising the steps of:
obtaining a plurality of projection exposures of an examination subject and electronically storing said plurality of projection exposures;
reconstructing slice images from said projection exposure using an image reconstruction technique;
manually medically evaluating said slice images and, in any of said slice images that is manually evaluated as containing a positive medical finding, marking the manually evaluated positive medical finding in the slice image with a first marker;
in a CAD system, automatically electronically medically evaluating said slice images and, in any of said slice images that is automatically electronically evaluated as containing a positive medical finding, marking a position of the automatically evaluated positive medical finding in the slice image with a second marker; and
automatically electronically comparing the slice images that were manually evaluated with the slice images that were automatically electronically evaluated and, from among slice images containing both a first marker and a second marker, identifying any slice images in which the position of the first marker deviates from the position of the second marker, and manually re-evaluating the identified slice images.

2. A method as claimed in claim 1 comprising employing a tomosynthesis technique as said reconstruction technique.

3. A method as claimed in claim 1 comprising, in said CAD system, automatically electronically evaluating said slice images by correlating adjacent slice images in said plurality of slice images.

4. A method as claimed in claim 1 comprising, in said CAD system, automatically electronically evaluating said slice images using only said projection exposures.

5. A method as claimed in claim 1 comprising, in said CAD system, automatically electronically evaluating said slice images using correlations between said slice images and said projection exposures.

6. A method as claimed in claim 1 comprising reconstructing said slice image in a form conforming to a classical mammography exposure, at least for use in said manual evaluation of said slice images.

7. A method as claimed in claim 1 comprising reconstructing said slice images with said reconstruction technique in a computer, said computer also comprising said CAD system.

8. A method as claimed in claim 1 comprising manually medically evaluating said projection exposures together with said slice images and, in said CAD system, automatically electronically medically evaluating said projection exposures together with said slice images.

9. A method as claimed in claim 1 comprising, with said reconstruction technique, reconstructing said slice images with respectively differing slice thicknesses.

10. A method as claimed in claim 1 comprising manually medically evaluating only selected ones of said slice images, and in said CAD system, automatically electronically medically evaluating all of said slice images.

11. A method as claimed in claim 1 wherein said slice images include a slice image of greater thickness, and comprising manually medically evaluating only said slice image of greater thickness and, in said CAD system automatically electronically medically evaluating all of said slice images.
